(19) United States
(12) Patent Application Publication
(10) Pub. No.: US 2013/0303496 A1
(43) Pub. Date: Nov. 14, 2013

(54) Method Of Treating Vascular Lesions

(71) Applicants: Daniel L. Cox, Palo Alto, CA (US); Stephen D. Pacetti, San Jose, CA (US)

(72) Inventors: Daniel L. Cox, Palo Alto, CA (US); Stephen D. Pacetti, San Jose, CA (US)

(73) Assignee: Abbott Cardiovascular Systems Inc., Santa Clara, CA (US)

(21) Appl. No.: 13/466,504

(22) Filed: May 8, 2012

(51) Int. Cl. A61K 31/573; A61P 7/00

(52) U.S. Cl. 514/171

(57) ABSTRACT

The present invention relates to the treatment of vascular lesions using low doses of an mTOR inhibitor together with a low dose of a glucocorticoid on an implantable medical device, wherein the treatment is particularly beneficial when the patient is a diabetic or is suffering from an abnormally long or diffuse lesion.

### FIELD

The present invention relates to an improved method of treating vascular lesions to facilitate healing especially in compromised patients such as diabetics and those suffering from particularly long or diffuse lesions or both. The method involves administration of a low dose of rapamycin or a derivative thereof together with a low dose of a glucocorticoid by means of an implantable medical device. The method will also be applicable to lesions in very small vessels.

### BACKGROUND

Until the mid-1980s, the accepted treatment for coronary atherosclerosis, i.e., narrowing of the coronary artery(ies) was coronary by-pass surgery. While being quite effective and having evolved to a relatively high degree of safety for such an invasive procedure, by-pass surgery still involves potentially serious complications and, in the best of cases, an extended recovery period.

With the advent of percutaneous transluminal coronary angioplasty (PTCA) in 1977, the scene changed dramatically. Using catheter techniques originally developed for heart exploration, inflatable balloons were deployed to re-open occluded regions in arteries. The procedure was relatively non-invasive, took a very short time compared to by-pass surgery and the recovery time was minimal. However, PTCA brought with it other problems such as vasospasm and elastic recoil of the stretched arterial wall which could undo much of what was accomplished and, in addition, engendered a new problem, restenosis, the re-clogging of the treated artery due to neointimal hyperplasia.

The next improvement, advanced in the mid-1980s, was the use of a stent to maintain luminal diameter after being re-established using PTCA. This for all intents and purposes put an end to vasospasm and elastic recoil but did not resolve the issue of restenosis. That is, prior to the introduction of stents, restenosis occurred in from about 30 to 50% of patients undergoing PTCA. Stenting reduced this to about 15 to 20%, a substantial improvement but still more than desirable.

In 2003, the drug-eluting stent (DES) was introduced. The drugs initially used with DESs were cytostatic compounds, that is, compounds that curtailed the proliferation of cells that resulted in restenosis. The occurrence of restenosis was reduced to about 5 to 7%, a relatively acceptable figure. However, the use of DESs engendered yet another complication, late stent thrombosis, the forming of blood clots long after the stent was in place. It was hypothesized that the formation of blood clots was most likely due to delayed healing, a side-effect of the use of cytostatic drugs.

It was found that the physiopathology of restenosis involves early injury to smooth muscle cells (SMCs), endothelial denudation and thrombus deposition. Over time, this leads to SMC proliferation and migration and extra-cellular matrix deposition. There is an increasing body of evidence suggesting that inflammation plays a pivotal role in linking this early vascular injury with neointimal growth and eventual lumen compromise, i.e., restenosis. Further, it has been observed that, when stents are used, the inflammatory state is often more intense and prolonged, exacerbating the situation.

To deal with the above, the dual-drug DES was developed. The dual drug DES carried an anti-proliferative drug to combat SMC proliferation and an anti-inflammatory drug to reduce inflammation. A particularly noteworthy family of anti-proliferative drugs is the mammalian target of rapamycin (mTOR) inhibitor family. mTOR inhibitors mitigate restenosis through inhibition of smooth muscle cell growth. mTOR inhibitors are, however, non-specific and also inhibit the growth of endothelial cells, which can slow the overall healing process, which may be implicated in late stent thrombosis.

Inflammation is, of course, a normal response to injury and is necessary for the healing process. However, chronic inflammation can be detrimental to healing in that the constant recruitment of monocytes, lymphocytes and neutrophils leads to a constant generation of inflammatory cytokines along with reactive oxygen species and enzymes generated by inflammatory cells to remove foreign bodies or damaged tissue. Thus, anti-inflammatory drugs are included in dual drug DESs to control chronic inflammation by reducing cytokine-driven neotintimal growth. Long-term administration of anti-inflammatory drugs, however, can also interfere with the healing process.

While generally quite effective, certain patient groups have not been completely served by current single-drug DESs. For example, in the SIRIUS clinical trial, patients with diabetes were roughly twice as likely as non-diabetics to incur binary restenosis. For lesions where the stents were well sized, diabetics exhibited restenosis in as high as 7.6% of cases for 20 mm lesions. In a study more indicative of routine clinical practice, the restenosis rate for long lesions, those greater than 40 mm in length, was 17.4%. Since DESs are being used to stent longer and longer lesions, these restenosis rates continues to pose a significant problem.

Vascular lesions in diabetic patients are considered difficult to manage for a number of reasons. The vasculature of diabetics is often in a state of chronic inflammation compared with those of non-diabetic patients. Further, in diabetics with chronic elevation of blood glucose levels, endothelial cells lining the blood vessels take in more glucose than normal, resulting in higher levels of surface glycoproteins. The basement membrane of the vessels then becomes thicker, weaker and more susceptible to lesions.

Diabetics are more prone to have what are termed diffuse lesions, as opposed to focal lesions. With simple, focal lesions, the region of stenosis has clear margins and is bounded by what are termed healthy reference vessel sections. In revascularizing a focal lesion, the objective is to dilate it such that the lumen matches that of the healthy reference vessel sections. Diffuse lesions have no such clear margins. They can be very long , and involve major sections of entire coronary arteries. Within the diffuse lesion, the lumen can vary widely in size with the distinction that none of it appears healthy, or is of a normal diameter. In treating diffuse lesions, the physician is posed with the dilemma of choosing a target lumen size since there is no clear healthy reference section. The physician then chooses a dilatation or stent diameter based on experience or the size of more distant section of coronary anatomy. Another challenge with diffuse lesions is determining how long of a vessel section to treat and this is also done based on experience. The long stents often required to treat diffuse lesions themselves come with a higher restenosis rate.

In addition, other vessels can be damaged in diabetics. For example, cardiomyopathy, nephropathy, neuropathy, retinopathy and foot nerve pain have all been found in diabetics with microvascular disease. Microvascular disease resulting from diabetes can also include inability to properly control blood flow due to damage to the endothelium's ability to relax and dilate.

What is needed is a method of treating vascular lesions that responds to the above concerns. This invention provides a method that not only will provide a significant improvement in treating vascular lesions generally but will be particularly useful for the treatment of diabetics and those with long or diffuse vascular lesions.

### SUMMARY

Thus, an aspect of this invention is a method of treating a vascular lesion in a patient, comprising delivering to the site of the vascular lesion an implantable medical device comprising a drug reservoir layer comprising about 20 to less than 100 μg/cm² of an mTOR inhibitor and about 40 μg/cm² to less than 200 μg/cm² of a glucocorticoid, wherein the release rate of both the mTOR inhibitor and the glucocorticoid is about 50% to about 90% at about 7 to about 90 days post implant.

In an aspect of this invention, the release rate of the mTOR inhibitor is about 50% to about 90% at about 28 days post implant.

In an aspect of this invention,the release rate of the glucocorticoid is about 60% to about 98% at about 28 days post implant.

In an aspect of this invention, the release rate of the mTOR inhibitor is about 80% at about 28 days post implant.

In an aspect of this invention, the release rate of the glucocorticoid is about 95% at about 28 days post implant.

In an aspect of this invention, the mTOR inhibitor is selected from the group consisting of everolimus, zotarolimus, sirolimus, sirolimus derivatives, biolimus, myolimus, novolimus, temsirolimus, merilimus, deforolimus and combinations thereof.

In an aspect of this invention, the mTOR inhibitor is zotarolimus.

In an aspect of this invention, the glucocorticoid is selected from the group consisting of dexamethasone and a derivative of dexamethasone that is as, or more, hydrophobic than dexamethasone.

In an aspect of this invention,the dexamethasone derivative is selected from the group consisting of dexamethasone acetate, dexamethasone laurate, dexamethasone tert-butylacetate, dexamethasone tetrahydrophthalate, and dexamethasone isonicotinate.

In an aspect of this invention, the glucocorticoid is dexamethasone acetate.

In an aspect of this invention,the implantable medical device comprises a stent.

In an aspect of this invention, the drug reservoir layer comprises a polymer or combination of polymers that exhibit a Hildebrand solubility parameter of about 7 to about 12.5 (cal/cm³)^{0.5}.

In an aspect of this invention, the polymer is selected from the group consisting of poly(vinylidene fluoride-co-hexafluoropropylene) (PVDF-HFP), poly(vinylidene fluoride) (PVDF), poly(vinylidene fluoride-co-chlorotrifluoroethylene) (PVDF-CTFE), poly(vinylidene fluoride-co-tetrafluoroethylene) (PVDF-TFE), poly(vinylidene fluoride-co-hexafluoropropylene-co-tetrafluoroethylene) and combinations thereof.

In an aspect of this invention, the vascular lesion is selected from the group consisting of diffuse or long lesions, small vessel lesions, saphenous vein graft lesions, restenotic lesions, bifurcation lesions, ostial lesions, left main lesions, chronic total occlusions and occlusions associated with AMI or STEMI.

In an aspect of this invention, the lesion is of the coronary, neurologic, carotid, aortic, renal, iliac, femoral, popliteal or tibial vasculature.

In an aspect of this invention, the drug reservoir layer comprises about 25 to about 75 μg/cm² of the mTOR inhibitor.

In an aspect of this invention, the drug reservoir layer comprises about 35 μg/cm² of zotarolimus.

In an aspect of this invention,the drug reservoir layer comprises about 50 to about 150 μg/cm² of the glucocortidoid.

In an aspect of this invention, the drug reservoir layer comprises about 70 μg/cm² of dexamethasone acetate.

In an aspect of this invention, the patient is a diabetic.

In an aspect of this invention,the vascular lesion is about 18 mm in length or longer.

In an aspect of this invention, the lesion is diffuse.

### DETAILED DESCRIPTION

### Brief Description of the Tables

Table 1 tabulates the composition of the test arms for porcine coronary safety evaluation using the method of this invention.

Table 2 tabulates the results of a histological comparison of inflammatory response to zotarolimus:dexamethasone-eluting Vision® and control stents at 28 days.

Table 3 tabulates the results of a histological comparison of inflammatory response to zotarolimus:dexamethasone-eluting Vision® and control stents at 90 days.

Table 4 tabulates the results of a morphometic comparison of cross-sectional vessel areas and neointimal response to zotarolimus:dexamethasone-eluting Vision® stents at 28 days.

Table 5 tabulates the results of a histological comparison of vessel injury and healing for zotarolimus:dexamethsone-eluting Vision® and control stents.

### Discussion

It is understood that use of the singular throughout this application including the claims includes the plural and vice versa unless expressly stated otherwise. That is, “a” and “the” are to be construed as referring to one or more of whatever the word modifies. Non-limiting examples are: “a therapeutic agent,” which is understood to include one or more such agents, and “a drug reservoir layer,” which is understood to include one or more such layers, unless it is expressly stated or is unambiguously obvious from the context that such is not intended.

As used herein, words of approximation such as, without limitation, “about,” “substantially,” “essentially” and “approximately” mean that the word or phrase modified by the term need not be exactly that which is written but may vary from that written description to some extent. The extent to which the description may vary will depend on how great a change can be instituted and have one of ordinary skill in the art recognize the modified version as still having the properties, characteristics and capabilities of the modified word or phrase. In general, but subject to the preceding discussion, a numerical value herein that is modified by a word of approximation may vary from the stated value by at least ±15%.

As used herein, the use of “preferred,” “preferably,” or “more preferred,” and the like refer to preferences as they existed at the time of filing of the patent application.

As used herein, “optional” means that the element modified by the term may, but is not required to, be present.

As used herein, “drug” and “therapeutic agent” are interchangeable and refer to a pharmacological substance use to treat a disease or disorder.

Treatment of “difficult to manage” (DTM) vascular lesions in diabetics has been slow in developing even though the restenosis rate in diabetics is currently in double digits, especially for longer lesions, while for non-diabetic patients and for simpler lesions lesion revascularization rate can be as low as 1.8%. It is currently estimated by the Center for Disease Control and Prevention (CDC) that one in ten Americans has diabetes in some form. The prediction for the future is not encouraging: the CDC predicts that by 2050, one in three Americans will have diabetes. While efforts are being made to lower these numbers by lifestyle and dietary changes, most likely such efforts will have a limited impact. Due to the large fraction of the general populace already afflicted with diabetes and the prediction of an even higher proportion in the future, treatments directed toward diabetics is much needed.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, and cochlear implants; prostheses, vascular grafts, self-expandable stents, balloon-expandable stents, stent-grafts, grafts, artificial heart valves, patent foramen ovale closure devices, left atrial appendage excluders, and cerebrospinal fluid shunts.

As used herein, “device body” refers to a fully formed implantable medical device with an outer surface to which no coating or layer of material different from that of which the device itself is manufactured has been applied. By “outer surface” is meant any surface however spatially oriented that is in contact with bodily tissue or fluids. A common example of a “device body” is a BMS, i.e., a bare metal stent, which is a fully-formed usable stent that has not been coated on any surface that is in contact with bodily tissue or fluids, with a layer of any material different from the metal of which it is made. “Device body” refers not only to BMSs but to any uncoated device regardless of what it is made of.

Presently preferred implantable medical devices of this invention are stents. A stent refers generally to any device used to hold tissue in place in a patient's body. Very often, stents are employed for the localized delivery of therapeutic agents to one or more specific treatment sites in a patient's body. Particularly useful stents are those used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an artery thought to be caused by inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, neuro, carotid, coronary, pulmonary, aorta, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

A stent used for patency maintenance is usually delivered to the target site in a compressed state and then expanded to fit the vessel into which it has been inserted. Once at a target location, a stent may be self-expandable or balloon expandable.

As used herein, a “primer layer” refers to a coating consisting of a polymer or blend of polymers that exhibit good adhesion characteristics with regard to the material of which the device body is manufactured and good adhesion characteristics with regard to whatever material is to be coated on the device body. Thus, a primer layer serves as an intermediary layer between a device body and materials to be affixed to the device body and is, therefore, applied directly to the device body. Examples of primers, without limitation, include acrylate and methacrylate polymers with poly(n-butyl methacrylate) (PBMA) being a presently preferred primer. Some additional examples of primers include, but are not limited to, poly(ethylene-co-vinyl alcohol), poly(vinyl acetate-co-vinyl alcohol), poly(methacrylates), poly(acrylates), polyethyleneamine, polyallylamine, chitosan, poly(ethylene-co-vinyl acetate), and parylene-C.

As use herein, a material that is described as a layer “disposed over” an indicated substrate, e.g., without limitation, a device body or another layer, refers to a relatively thin coating of the material applied, preferably at present, directly to essentially the entire exposed surface of the indicated substrate. By “exposed surface” is meant any surface regardless of its physical location with respect to the configuration of the device that, in use, would be in contact with bodily tissues or fluids. “Disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “drug reservoir layer” refers either to a layer of one or more therapeutic agents applied neat or as a layer of polymer or blend of polymers that has dispersed within its three-dimensional structure one or more therapeutic agents. A polymeric drug reservoir layer is designed such that, by one mechanism or another, e.g., without limitation, by elution or as the result of biodegradation of the polymer, the therapeutic substance is released from the layer into the surrounding environment. For the purpose of this invention, the drug reservoir layer also acts as rate-controlling layer. As used herein, “rate-controlling layer” refers to a polymer layer that controls the release of therapeutic agents or drugs into the environment.

As used herein, “therapeutic agent” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a therapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, the terms “drug” and “therapeutic agent” are used interchangeably.

As used herein, “treating” refers to the administration of a therapeutically effective amount of a therapeutic agent to a patient known or suspected to be afflicted with a vascular disease.

A “therapeutically effective amount” refers to that amount of a therapeutic agent that will have a beneficial effect, which may be curative or palliative, on the health and well-being of the patient with regard to the vascular disease with which the patient is known or suspected to be afflicted. A therapeutically effective amount may be administered as a single bolus, as intermittent bolus charges, as short, medium or long term sustained release formulations or as any combination of these. As used herein, short-term sustained release refers to the administration of a therapeutically effective amount of a therapeutic agent over a period from about several hours to about 3 days. Medium-term sustained release refers to administration of a therapeutically effective amount of a therapeutic agent over a period from about 3 day to about 14 days and long-term refers to the delivery of a therapeutically effective amount over any period in excess of about 14 days. Presently it is preferred to deliver a therapeutically effective amount of a drug for a period of about 7 days to a period of about 28 days, although longer durations are also included.

As used herein, a “patient” refers to any living organism that might benefit from the application of the implantable medical device and method of this invention. Preferably the patient is a mammal and most preferably at present the patient is a human being.

As used herein, a “vascular disease” refers to a disease of the vessels, primarily arteries and veins, which transport blood to and from the heart, brain and peripheral organs such as, without limitation, the arms, legs, kidneys and liver. In particular “vascular disease” refers to the coronary arterial and venous systems, the carotid arterial and venous systems, the aortic arterial and venous systems and the peripheral arterial and venous systems. The disease that may be treated is any that is amenable to treatment with a therapeutic agent, either as the sole treatment protocol or as an adjunct to other procedures such as surgical intervention. The disease may be, without limitation, atherosclerosis, vulnerable plaque, restenosis or peripheral arterial disease. Peripheral vascular disease includes arterial and venous diseases of the renal, iliac, femoral, popliteal, tibial and other vascular regions.

Peripheral vascular diseases are generally caused by structural changes in blood vessels caused by such conditions as inflammation and tissue damage. A subset of peripheral vascular disease is peripheral artery disease (PAD). PAD is a condition that is similar to carotid and coronary artery disease in that it is caused by the buildup of fatty deposits on the lining or intima of the artery walls. Just as blockage of the carotid artery restricts blood flow to the brain and blockage of the coronary artery restricts blood flow to the heart, blockage of the peripheral arteries can lead to restricted blood flow to the kidneys, stomach, arms, legs and feet. In particular at present a peripheral vascular disease often refers to a vascular disease of the superficial femoral artery.

As used herein, a “vascular lesion” refers to a vascular disease involving localized pathological change in the vasculature, in particular a change that results in compromising the patency of the vasculature in the vicinity of the lesion. Examples of vascular lesions include, without limitation, saphenous vein graft lesions, de novo lesions, small vessel lesions, restenotic lesions, bifurcation lesions, ostial lesions, left main lesions, chronic total occlusions and occlusions associated with AMI (Acute Myocardial Infarction), STEMI (ST Segment Elevation Myocardial Infarction) or non-STEMI (non-ST Segment Elevation Myocardial Infarction).

As used herein, a DTM refers to a lesion for which standard treatment protocols have proven less effective or more prone to undesirable side effects or both. Such lesions include, without limitation, those of diabetic patients in particular, it being widely known that diabetics tend to present with more complex coronary lesions and also tend to be more challenging to treat due to various diabetic complications. Further, a DTM vascular lesion refers to lesions that by virtue of their physical characteristics such as, without limitation, diffusivity or abnormal length, that is, lesions that are about 18 mm or longer, do not respond well to standard treatment protocols. Finally, a vascular lesion in a particularly small vessel such as, without limitation, those less that 2.5 mm in diameter, constitutes a DTM vascular lesion within the scope of this invention.

A DTM vascular lesion may occur in any vascular region including, without limitation, arteries and veins in the carotid, aortic, renal, iliac, femoral, popliteal and tibial vasculature.

For the purposes of this invention, a DTM vascular lesion is considered to be a vascular disease.

“Atherosclerosis” refers to the depositing of fatty substances, cholesterol, cellular waste products, calcium and fibrin on the inner lining or intima of an artery. Smooth muscle cell proliferation and lipid accumulation accompany the deposition process. In addition, inflammatory substances that tend to migrate to atherosclerotic regions of an artery are thought to exacerbate the condition. The result of the accumulation of substances on the intima is the formation of fibrous (atheromatous) plaques that occlude the lumen of the artery, a process called stenosis. When the stenosis becomes severe enough, the blood supply to the organ supplied by the particular artery is depleted resulting in a stroke, if the afflicted artery is a carotid artery, heart attack if the artery is coronary, or loss of organ or limb function if the artery is peripheral.

“Restenosis” refers to the re-narrowing of an artery at or near the site where angioplasty or another surgical procedure was previously performed to remove a stenosis. It is generally due to smooth muscle cell proliferation and, at times, is accompanied by thrombosis. Prior to the advent of implantable stents to maintain the patency of vessels opened by angioplasty, restenosis occurred in 40-50% of patients within 3 to 6 months of undergoing the procedure. Post-angioplasty restenosis before stents was due primarily to smooth muscle cell proliferation. However, there were also issues of acute re-closure due to vasospasm, dissection, and thrombosis at the site of the procedure. Stents eliminated acute closure from vasospasm and greatly reduced complications from dissections. The use of Ilb-Ills anti-platelet drugs such as abciximab and epifabatide, and anti-platelet agents such as ticlopidine, clopidogrel, prasugrel and ticagrelor, which are anti-thrombotic, reduced the occurrence of post-procedure clotting. Stent placement sites are also susceptible to restenosis due to abnormal tissue growth at the site of implantation. This form of restenosis tends also to occur at 3 to 6 months after stent placement but it is not affected by the use of anti-clotting drugs. Thus, alternative therapies are continuously being sought to mitigate, preferably eliminate, this type of restenosis. Drug eluting stents (DES) which release a variety of therapeutic agents at the site of stent placement have been in use for some time. To date, these coronary stents comprise drug delivery surfaces (lengths) that are typically less than 40 mm in length and have delivery surfaces that are not intended, and most often do not, contact the luminal surface of the vessel at the non-afflicted regions at the periphery of the afflicted region.

“Vulnerable plaque” refers to an atheromatous plaque that has the potential of causing a thrombotic event and is usually characterized by a thin fibrous cap separating a lipid filled atheroma from the lumen of an artery. The thinness of the cap renders the plaque susceptible to rupture. When the plaque ruptures, the inner core of usually lipid-rich plaque is exposed to blood. This releases tissue factor and lipid components with the potential of causing a potentially fatal thrombotic event through adhesion and activation of platelets and plasma proteins to components of the exposed plaque.

The phenomenon of “vulnerable plaque” has created new challenges in recent years for the treatment of heart disease. Unlike occlusive plaques that impede blood flow, vulnerable plaque develops within the arterial walls, and in its early stages does so without the characteristic substantial narrowing of the arterial lumen which produces symptoms. As such, conventional methods for detecting heart disease, such as an angiogram, may not detect vulnerable plaque growth into the arterial wall.

“Thrombosis” refers to the formation or presence of a blood clot (thrombus) inside a blood vessel or chamber of the heart. A blood clot that breaks off and travels to another part of the body is called an embolus. If a clot blocks a blood vessel that feeds the heart, it causes a heart attack. If a clot blocks a blood vessel that feeds to brain, it causes a stroke.

As used herein, “eluting” as relating to a therapeutic agent from a drug reservoir layer of this invention refers to the exodus of the drug, and potentially other therapeutic agents, from the drug reservoir layer into the surrounding environment. The “surrounding environment” ordinarily will constitute the lumen of a vessel or the wall of that lumen which in turn may mean directly into the cells forming the wall or into the intercellular space.

It is presently preferred that a drug reservoir layer polymer of this invention have a Hildebrand solubility parameter of about 7 to about 12.5 (cal/cm³)^{0.5} Suitable polymers include, without limitation, poly(vinylidene fluoride) (PVDF), poly(vinylidene fluoride-co-hexafluoropropylene) (PVDF-HFP), poly(vinylidene fluoride-co-chlorotrifluoroethylene) (PVDF-CTFE), poly(vinylidene fluoride-co-hexafluoropropylene-co-tetrafluoroethylene), poly(vinylidene fluoride-co-tetrafluoroethylene) (PVDF-TFE), and combinations thereof. It is presently preferred that the polymer have at least 25% vinylidene fluoride by weight. For the purposes of this invention a vinylidene fluoride containing polymer having a weight average molecular weight of from about 40,000 to about 750,000 Daltons is presently preferred. To function optimally as a stent coating, a polymer must satisfy several criteria. Vinylidene fluoride based polymers can have both good elongation properties to accommodate stent expansion, as well as good toughness to withstand the rigors of stent crimping and delivery to a lesion site. This family of polymers has, in general, a sub-ambient glass transition temperature and can be formulated to provide for controlled drug release. They are very stable polymers due to a polymer backbone of only carbon-carbon bonds with all pendant bonds being either C—H or C—F. This confers great chemical stability during processing and in vivo. The long-term biocompatibility tends to be good for this class of polymers due to their purity and lack or reactivity. In addition, fluorinated surfaces provide good thrombo-resistance/hemocompatibility.

The therapeutic agents herein are contained in the polymeric drug reservoir layer. They are delivered to the site where needed by implantation of the medical device into the patient. Therapeutic agents that may be used in the present invention include, without limitation, antiproliferative agents, anti-inflammatory agents, antineoplastics, antimitotics, antiplatelet, anticoagulant, antifibrin, and antithrombin drugs, cytostatic or antiproliferative agents, antibiotics, antiallergic agents and antioxidants.

Presently preferred is the use of an antiproliferative agent combined with an anti-inflammatory agent.

Suitable antiproliferative agents that can be used in the present invention include, without limitation, mTOR inhibitors, actinomycin D, taxol, docetaxel, paclitaxel, FKBP-12 mediated mTOR inhibitors, perfenidone and prodrugs, co-drugs and combinations thereof.

Presently preferred mTOR inhibitors include everolimus, zotarolimus, sirolimus, sirolimus derivatives, biolimus, myolimus, novolimus, temsirolimus, merilimus, deforolimus and combinations thereof. Zotarolimus is presently a preferred mTOR inhibitor for use in the method of this invention. Zotarolimus is a semi-synthetic derivative of rapamycin, a naturally product isolated from Streptomyces hydroscopicus, and is prepared by substituting a tetrazole moiety for the hydroxyl group at position 42 of rapamycin. Zotarolimus is extremely lipophilic, which is an advantageous property with regard to delivery of the compound from a drug reservoir layer of a stent. The compound's hydrophobicity permits slow sustained release from a hydrophobic polymer, which in turn facilitates maintenance of therapeutic drug levels eluting from the drug reservoir layer of the stent. This very low water solubility also leads to a long residence time in tissues. Further, its lipophilic character favors crossing of cell membranes to inhibit neointimal proliferation of target tissues.

The dose density of an anti-proliferative drug in a drug reservoir layer of this invention is about 10 μg/cm² to about 1000 μg/cm², preferably about 50 μg/cm² to about 500 μg/cm² and even more preferably about 20 μg/cm² to about 100 μg/cm² of stent surface area. In particular, when the anti-proliferative is a mTOR inhibitor, the dose density is preferably about 25 μg/cc² to about 75 μg/cm² of stent surface area and when the mTOR inhibitor is zotarolimus, the presently preferred dose is about 35 μg/cm² of stent surface area.

Suitable anti-inflammatory agents that can be used in combination with the antiproliferative(s) include, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus and prodrugs, co-drugs and combinations thereof.

Presently preferred anti-inflammatory drugs for use in the present invention are glucocorticoids, such as dexamethasone or derivatives thereof that are as or more hydrophobic than dexamethasone itself. Examples include, without limitation, dexamethasone acetate, dexamethasone laurate, dexamethasone-tert-butylacetate, dexamethasone tetrahydrophthalate, and dexamethasone isonicotinate. The presently preferred dexamethasone derivative is dexamethasone acetate.

The amount of the dexamethasone or derivative thereof in a drug reservoir layer of this invention is from about 40 μg/cm² to about 200 μg/cm² of stent surface area, preferably between about 50 μg/cm² to about 100 μg/cm² of stent surface area, and presently, when the derivative is dexamethasone acetate, most preferably about 70 μg/cm² of stent surface area.

Sustained release of the anti-proliferative and anti-inflammatory drugs of this invention will occur over a period of about 7 to about 90 days and, when the anti-proliferative is an mTOR inhibitor and the anti-inflammatory is a glucocorticoid, preferably over 7 to 28 days.

The release rate of the anti-proliferative and anti-inflammatory drugs from an implantable medical device will be about 50% to about 90% over the indicated time period. With an mTOR anti-proliferative and a glucocorticoid anti-inflammatory drug the presently preferred release rate is about 50% to about 90% over 28 days, most preferably at present about 80% over 28 days.

For treatment of DTM vascular lesions using the method of this invention, the drug doses are minimized to prevent or at least ameliorate any negative effect on healing. The dose of the antiproliferative drug is, of course, calculated to still be sufficient to treat the patient's vascular lesion(s) by inhibiting proliferation of smooth muscle cells, which could otherwise lead to restenosis. By lowering the dose of the antiproliferative the inhibitory effect on endothelial cell proliferation is reduced. By itself, the anti-inflammatory drug does not typically inhibit smooth muscle cell proliferation. However, when combined with the antiproliferative drug, a synergistic effect is observed where the inhibition of neointimal growth is greater with a given dose of antiproliferative plus anti-inflammatory compared to that achieved with the antiproliferative or the anti-inflammatory alone.

For revascularization using a drug-eluting stent, the most important aspect of healing is re-endothelialization of the treated segment where the endothelium is not only complete, but also functional. High doses of antiproliferative drug and, in particular, high doses of antiproliferative drug plus high doses of anti-inflammatory drug can inhibit this healing. The drug release rate, however, also has an impact healing. For a given dose of drug, a longer duration of drug release has a greater inhibition of both neointimal proliferation and healing compared with a shorter duration of drug release.

Achieving a balance between improved efficacy in treatment of vascular lesions, in particular DTM vascular lesions, while maintaining or improving the safety of the procedure requires careful selection of drug doses and release rates. A dose that is too low, or a release rate that is too fast, may not achieve the desired inhibition of neointimal growth. Conversely, a dose that is too high or a release rate that is too slow may inhibit healing of the vessel and the formation of a functional endothelium. This invention provides an optimal balance of all parameters to treat DTM vascular lesions.

### Materials and Methods

For the following experiments, zotarolimus was provided by ScinoPharm. Dexamethasone acetate was provided by AKSci. Vision® stents were obtained from Abbott Vascular. These were bare metal stents measuring 3.0 mm by 12 mm. Preclinical studies in a porcine model were performed at Synecor and the subsequent tissue processing and histological analyses were carried out at CVPath Institute, Inc. Tissue sections were stained with hematoxylin and eosin, and Van Gieson stains.

### Porcine Implant Studies

To study the effect of dose and release rate for an mTOR inhibitor and a glucocorticoid, drug-eluting stents containing a range of doses of zotarolimus and dexamethasone acetate and a range of release rates were prepared. These dual-drug eluting stents were evaluated in a porcine coronary efficacy and vascular response study. All arms used 3.0 mm×12 mm Vision® Rx balloon coronary stent delivery systems. All stents were first coated with a primer layer of poly(n-butyl methacrylate) (PBMA). A combination of zotarolimus and dexamethasone acetate in PVDF-HFP polymer was then applied over the primer to form the drug reservoir layer. After mounting on the delivery catheter, the units were sterilized by ethylene oxide. The total drug dose was varied by altering the drug/polymer ratio and the total coating weight. These same parameters were also utilized to adjust the drug release rates. The target release rates of the zotarolimus are shown in Table 1. The stents were implanted in domestic farm swine at a 1.1:1 overstretch ratio, and both 28 day and 90 day time points were studied. One stent was implanted in each of the three coronary arteries, and all pigs had a control everolimus-eluting Vision® coronary stent implanted into one of the coronary arteries. Table 1 provides the composition of the test arms for use in the porcine safety evaluation.

TABLE 1

| | Stents/ |
|---|---|
| Description | Timepoint |
| Arm 1 - 35:70 μg/cm², RR = ~80% at 28 days | 12 |
| Arm 2 - 35:70 μg/cm², RR = ~80% at 7 days | 12 |
| Arm 3 - 35:140 μg/cm², RR = ~80% at 28 days | 12 |
| Arm 4 - 35:140 μg/cm², RR = ~80% at 7 days | 12 |
| Arm 5 - 100:200 μg/cm², RR = ~80% at 28 days | 12 |
| Arm 6 - 20:40 μg/cm², RR = ~80% at 1 day | 12 |
| Arm 7 - Everolimus-eluting Vision ^{®} coronary | 37 |
| stent with PVDF-HFP reservoir layer, 100 μg/cm², | |
| RR = ~80% @ 28 days (control) | |

In Table 1, the first number is the zotarolimus dose and the second number is the dexamethasone acetate dose in micrograms of drug per square centimeter of stent surface area. RR is the targeted release rate for the zotarolimus. Dose and release rates for the zotarolimus were tuned by adjusting the drug/polymer ratio and the total coating weight. The dexamethasone acetate release rate followed similar trends in all arms but released faster, primarily due to its lower molecular weight and higher diffusivity in the polymer. Thus, Arm 1 has 35 μg of zotarolimus and 70 μg of dexamethasone acetate per square centimeter of polymer. The desired release rate for Arm 1 was about 80% release of zotarolimus at 28 days after implantation of the stents in the animals. Arm 2 had the same ratio of dexamethasone acetate to zotarolimus, but a higher drug to polymer ratio for both drugs. This resulted in a release rate of about 80% of zotarolimus at 7 days after implantation. There were 12 stents for each of the arms except for Arm 7, which had 37 stents implanted. Implantation times were either 28 or 90 days and are noted in the data tables below. For example, in Table 2, even though Arms 2 and 4 used stents that released 80% of the zotarolimus at 7 days, the stents were not removed until 28 days after implantation.

The drug doses and release rates shown in Table 1 were chosen to represent a broad range that would still be practical to manufacture. Given the expected relative effects of the drugs, the dexamethasone acetate was always present at a higher dose than the zotarolimus because dexamethasone acetate has been shown in cell culture studies to exhibit a lower potency drug than zotarolimus for the control of proliferation and because dexamethasone acetate releases faster from the drug reservoir layer. The lowest dose of zotarolimus was based on what was estimated to be capable of being manufactured under medical device quality guidelines. Analytical methods for measuring drug impurities have limits of quantitation which can be reached for small stents with low drug dosages. The highest zotarolimus dose was selected to match an everolimus dose used on a commercial DES. The highest dexamethasone acetate dose was limited in order to control the drug coating thickness. The slowest drug release rate was based on the drug release rates of effective, commercial DES. A one day release target for the low dose was used to try to find a limit where efficacy was not seen in this animal model. The intermediate doses and release rates were chosen to examine the interplay between neointimal inhibition and vascular healing.

The results shown in Table 2 demonstrate a dose and release rate window with well-defined boundaries for both safety and efficacy. Table 2 shows a histological comparisons at 28 days of inflammatory response to zotarolimus:dexamethasone-eluting Vision® stents and control stents.

TABLE 2

| | | Intimal | Adventitial | |
|---|---|---|---|---|
| Treat- | | Inflam- | Inflam- | |
| ment | Granulomas | mation | mation | Giant Cells |
| Group | (%) | Score | Score | (%) |
| Arm 1 | 0 ± 0 | 0.18 ± 0.60 | 0 ± 0 | 4.15 ± 12.25 |
| (n = 11) | | | | |
| Arm 2 | 0.78 ± 1.66 | 0.50 ± 0.98 | 0.067 ± 0.21 | 10.38 ± 17.00 |
| (n = 10) | | | | |
| Arm 3 | 0 ± 0 | 0.55 ± 0.96 | 0 ± 0 | 6.65 ± 14.69 |
| (n = 11) | | | | |
| Arm 4 | 0 ± 0 | 0.70 ± 0.84 | 0.03 ± 0.10 | 11.21 ± 16.60 |
| (n = 11) | | | | |
| Arm 5 | 0 ± 0 | 0.19 ± 0.41 | 0 ± 0 | 1.86 ± 3.53 |
| (n = 12) | | | | |
| Arm 6 | 1.99 ± 3.62 | 0.53 ± 0.72 | 0.50 ± 0.67 | 6.94 ± 6.46 |
| (n = 12) | | | | |
| Arm 7 | 21.77 ± 30.86 | 1.58 ± 1.81 | 0.81 ± 0.96 | 20.04 ± 17.58 |
| (n = 37) | | | | |
| p-value | 0.0001-A1, | 0.116 | <0.0001* | 0.0018-A1, A5. |
| | A2, A3, A4, | | | A6 |
| | A5, A6 | | | v. |
| | v. | | | A7 control |
| | A7 control | | | |

The treatment arms in this table are defined in Table 1. In Table 2, samples were taken at 28 days after implantation. The value of “n” is the number of stents tested. Scoring was done based on the histopathology results. The intimal and adventitial inflammation scores are based on inflammation scores on just the neointima and the adventia lying outside of the media. “Giant cells (%)” was the percent of struts with giant cells.

One indication of the relative effect of the dose and release rate is the presence of granulomas. Granulomas are comprised of granulation tissue containing macrophages, lymphocytes and some eosinophils. A moderate level of inflammation and granulomas were observed in arm 7, an observation often made in porcine studies.

The presence of granulomas has been associated with increased neointima in the porcine model. The control Arm 7 had granulomas appearing in 21.77% of the struts. With the lowest dose/fastest release system of zotarolimus/ dexamethasone acetate (Arm 6), this dropped to only 1.99% of struts. The only other test arm with granulomas was Arm 2, which is the next lowest dose, with the next fastest drug release profile. This indicates that the combination of zotarolimus and dexamethasone acetate reduces the occurrence of granulomas in this model. The effect is reduced as the dexamethasone acetate dose is reduced and/or the release rate is increased. These data establish the range of dexamethasone acetate dosages and release rates that provide effective suppression of inflammation.

The effect on granulomas at 90 days is shown in Table 3 which tabulates a histological comparison of inflammatory response of zotarolimus:dexamethasone-eluting Vision® stents and control stents.

TABLE 3

| | | Intimal | Adventitial | | |
|---|---|---|---|---|---|
| Treatment | Granulomas | Inflammation | Inflammation | Giant Cells | Calcification |
| Group | (%) | Score | Score | (%) | (%) |
| Arm 1 | 0.20 ± 0.69 | 0.11 ± 0.22 | 0.25 ± 0.59 | 0.20 ± 0.69 | 9.42 ± 15.06 |
| (n = 12) | | | | | |
| Arm 2 | 53.61 ± 40.18 | 3.03 ± 1.59 | 1.19 ± 0.87 | 13.50 ± 11.66 | 0 ± 0 |
| (n = 12) | | | | | |
| Arm 3 | 0 ± 0 | 0.028 ± 0.96 | 0.028 ± 0.096 | 0 ± 0 | 12.32 ± 12.46 |
| (n = 12) | | | | | |
| Arm 4 | 53.86 ± 44.62 | 2.61 ± 1.79 | 0.97 ± 0.63 | 11.28 ± 12.89 | 2.62 ± 3.15 |
| (n = 12) | | | | | |
| Arm 5 | 0.56 ± 1.92 | 0.17 ± 0.58 | 0.11 ± 0.38 | 0 ± 0 | 7.73 ± 9.03 |
| (n = 12) | | | | | |
| Arm 6 | 43.39 ± 37.50 | 2.78 ± 1.75 | 0.89 ± 0.64 | 21.61 ± 16.10 | 1.99 ± 4.31 |
| (n = 12) | | | | | |
| Arm 7 | 53.78 ± 45.77 | 2.50 ± 1.79 | 0.75 ± 0.64 | 14.19 ± 16.06 | 2.13 ± 5.96 |
| (n = 37) | | | | | |
| p-value | <0.0001 | <0.0001* | <0.0001* | <0.0001 | 0.0006 |
| Arm1 v | S | 0.0002 | 0.0110 | S | S |
| Arm7 | | | | | |
| Arm2 v | | 0.406 | 0.0971 | | |
| Arm7 | | | | | |
| Arm3 v | S | <0.0001 | 0.0003 | S | S |
| Arm7 | | | | | |
| Arm4 v | | 0.899 | 0.318 | | |
| Arm7 | | | | | |
| Arm5 v | S | <0.0001 | 0.0012 | S | |
| Arm7 | | | | | |
| Arm6 v | | 0.600 | 0.585 | | |
| Arm7 | | | | | |

“S” indicates the groups are significantly different. Data for the treatment groups was obtained as for Table 2. Calcification percentage was also included, and represents the percentage of struts with calcium usually evidenced as dark specks or deposits

At 90 days, Arm 7 had 53.78% of struts with granulomas. In the porcine model, the inflammatory response typically peaks at 90 days, with resolution at longer time points. The lasting effect of the dual drug Zotarolimus and Dexamethasone system on granulomas is still seen, but only for the systems with a longer duration of drug release, that is, 28 days (Arms 1, 3 and 5). Arms 2 and 4 (7 day drug release) and 6 (1 day drug release) were very similar to Arm 7.

A key measure of efficacy is the intimal area seen at 28 days compared to controls. Table 4 shows a morphometric comparison at 28 days of cross-sectional vessel areas and neointimal response using zotarolimus:dexamethasone-eluting Vision® stents and control stents.

TABLE 4

| | | | | | | | Mean |
|---|---|---|---|---|---|---|---|
| | EEL | | Lumen | Intimal | Medial | | Intimal |
| Treatment | Area | IEL Area | Area | Area | Area | | Thickness |
| Group | (mm²) | (mm²) | (mm²) | (mm²) | (mm²) | Stenosis % | (mm) |
| Arm 1 | 7.67 ± 0.87 | 6.75 ± 0.76 | 6.12 ± 0.67 | 0.63 ± 0.18 | 0.92 ± 0.20 | 9.48 ± 2.43 | 0.030 ± 0.024 |
| (n = 11) | | | | | | | |
| Arm 2 | 8.52 ± 1.30 | 7.45 ± 1.18 | 6.58 ± 1.08 | 0.87 ± 0.20 | 1.07 ± 0.19 | 11.88 ± 2.25 | 0.053 ± 0.028 |
| (n = 10) | | | | | | | |
| Arm 3 | 7.53 ± 1.06 | 6.64 ± 0.99 | 5.96 ± 1.01 | 0.69 ± 0.20 | 0.89 ± 0.17 | 10.50 ± 3.26 | 0.035 ± 0.020 |
| (n = 11) | | | | | | | |
| Arm 4 | 8.30 ± 0.80 | 7.25 ± 0.72 | 6.38 ± 0.68 | 0.88 ± 0.31 | 1.04 ± 0.13 | 12.08 ± 3.93 | 0.052 ± 0.032 |
| (n = 11) | | | | | | | |
| Arm 5 | 7.85 ± 0.82 | 6.97 ± 0.73 | 6.40 ± 0.69 | 0.57 ± 0.091 | 0.88 ± 0.13 | 8.21 ± 1.23 | 0.022 ± 0.0068 |
| (n = 12) | | | | | | | |
| Arm 6 | 8.29 ± 1.01 | 7.10 ± 0.94 | 5.66 ± 1.12 | 1.44 ± 0.55 | 1.19 ± 0.18 | 20.80 ± 8.79 | 0.13 ± 0.079 |
| (n = 12) | | | | | | | |
| Arm 7 | 8.37 ± 1.24 | 6.86 ± 1.03 | 4.85 ± 1.63 | 2.01 ± 0.95 | 1.51 ± 0.54 | 30.83 ± 17.08 | 0.25 ± 0.20 |
| (n = 37) | | | | | | | |
| p-value | 0.155 | 0.408 | .0001- | .0001- | .0001- | .0001- | .0001- |
| | | | A1, A2, | A1, A2, | A1, A2, | A1, A2, | A1, A2, |
| | | | A4, A5 | A3, A4, | A3, A4, | A3, A4, | A3, A4, |
| | | | v. A7 | A5, A6 | A5, A6 | A5, A6 | A5, A6 |
| | | | | v. A7 | v. A7 | v. A7 | v. A7 |

Histomorphometric parameters are defined as follows: EEL area is the external elastic lamina area; IEL area is the internal elastic lamina area ; lumen area is the area where blood flows; intimal area is the internal elastic lamina area minus luminal area; medial area is the external elastic area minus internal elastic area; Stenosis is the percent area within the IEL which has become neointima (100×[1−(Lumen area/IEL)]); Mean Intimal Thickness is the average neointimal thickness in mm.

The intimal areas of all test groups (Arms 1-6) were statistically lower than the 2.01 mm² Intimal area of Arm 7. After the arm 7 control, the least efficacious test group was arm 6 with the lowest drug doses and fastest drug release. The percent stenosis and mean intimal thickness are also measures of efficacy and showed a similar effect. This indicates that in terms of efficacy, all arms containing dexamethasone acetate were more efficacious than the everolimus only control. Inflammation is a strong stimulus for neointimal proliferation and these data show that effect suppression of inflammation translates into high efficacy against neointimal proliferation. If a dexamethasone acetate only arm were present it would likely show very little, if any, efficacy.

Table 5 shows a histologic comparison after 28 days of vessel injury and healing for zotarolimus:dexamethasone-eluting Vision® stents and control stents.

TABLE 5

| | | | Mean | | | | Uncovered |
|---|---|---|---|---|---|---|---|
| Treatment | Injury | Fibrin | Fibrin | Malapposed | RBC | Endothelialization | Stents |
| Group | Score | (%) | Score | (%) | (%) | (%) | (%) |
| Arm 1 | 0.23 ± 0.21 | 86.89 ± 16.40 | 1.97 ± 0.35 | 0 ± 0 | 22.01 ± 16.61 | 98.91 ± 1.69 | 1.53 ± 3.10 |
| (n = 11) | | | | | | | |
| Arm 2 | 0.17 ± 0.16 | 85.10 ± 21.31 | 1.70 ± 0.48 | 0.37 ± 1.11 | 24.16 ± 15.63 | 99.41 ± 1.09 | 0.37 ± 1.11 |
| (n = 9) | | | | | | | |
| Arm 3 | 0.21 ± 0.11 | 90.37 ± 9.96 | 2.12 ± 0.62 | 0 ± 0 | 29.94 ± 16.32 | 98.12 ± 4.09 | 2.53 ± 6.38 |
| (n = 11) | | | | | | | |
| Arm 4 | 0.12 ± 0.11 | 82.33 ± 20.70 | 1.73 ± 0.33 | 0 ± 0 | 18.11 ± 12.51 | 99.61 ± 1.09 | 0.55 ± 1.83 |
| (n = 11) | | | | | | | |
| Arm 5 | 0.12 ± 0.083 | 70.97 ± 32.45 | 1.64 ± 0.77 | 0 ± 0 | 24.57 ± 17.80 | 85.17 ± 21.75 | 23.28 ± 32.04 |
| (n = 12) | | | | | | | |
| Arm 6 | 0.23 ± 0.18 | 84.00 ± 15.48 | 1.56 ± 0.37 | 0 ± 0 | 17.70 ± 14.88 | 99.96 ± 0.11 | 0 ± 0 |
| (n = 9) | | | | | | | |
| Arm 7 | 0.17 ± 0.14 | 93.92 ± 12.44 | 1.87 ± 0.31 | 0 ± 0 | 8.20 ± 11.64 | 99.89 ± 0.26 | 0 ± 0 |
| (control) | | | | | | | |
| (n = 20) | | | | | | | |
| p-value | 0.564 | 0.0802 | 0.0620 | 0.221 | 0.0061- | 0.0003- | <0.0001- |
| | | | | | A2, A3, A5 | A5 v A7 | A5 v A7 |
| | | | | | v A7 | | |

Percent endothelialization is an important measure of safety, and lack thereof, is an indicator of delayed or incomplete healing. Arm 7 (control), and all of the test arms showed nearly complete endothelialization except for the highest dose/longest release system, Arm 5. This group showed only 85% endothelialization at 28 days. It also showed far more uncovered struts than any other group. Arm 5 shows that a high dose/long release of both drugs results in healing that is less than that obtained using the everolimus-only control.

Thus, this invention provides a dose and release rate window with well-defined boundaries for both safety and efficacy to treat DTM vascular lesions. The combination of dexamethasone acetate at a 200 μg/cm² dose with a 100 μg/cm² dose of zotarolimus which released 80% at 28 days shows incomplete endothelialization at 28 days. This raises concerns about sacrificing healing, and possibly safety, in exchange for anti-restenotic efficacy. This puts an upper limit on the doses to be considered. The addition of dexamethasone acetate at a 40 μg/cm² dose to a 20 μg/cm² dose of zotarolimus of which 80% is released in 1 day shows some efficacy, but not the greater efficacy desired to treat, for example, a diabetic patient. The intermediate drug doses represented by arms 1 through 4 are more acceptable for efficacy. However, the more profound suppression of neointima, even in the presence of granulomas, is only present at 90 days for the slower release rate (Arms 1 and 3, which release 80% of the zotarolimus at 28 days). For the 35 μg/cm² dose of zotarolimus, there was no incremental benefit seen for the 140 μg/cm² dose of dexamethasone acetate compared to the 70 μg/cm² dose.

As can be seen, the best efficacy and safety result for treating a proposed DTM vascular lesion is 35 μg/cm² zotarolimus combined with 70 μg/cm² dexamethasone acetate with a release rate of 80% at 28 days for the zotarolimus.

It is expected that the method of this invention can be extended to other anti-proliferative drugs such as those listed previously herein. Dexamethasone acetate was selected for this study primarily due to its compatibility with the PVDF-HFP polymer. The method of this invention is expected to apply to other dexamethasone derivatives as well as other anti-inflammatory drugs. The polymer(s) of the drug reservoir layer may be different depending on the properties of the anti-proliferative and anti-inflammatory but the determination of such should be well within the ability of the skilled artisan based on the disclosure herein.

In summary, a tailored treatment of DTM vascular lesions pursuant to this invention is as follows:
A dual drug drug-eluting stent (DES) consisting of an mTOR inhibitor and a glucocorticoid;
An mTOR inhibitor selected from the group consisting of zotarolimus, everolimus, sirolimus, biolimus, myolimus, novolimus, tensirolimus, merolimus, deforolimus, other derivatives of sirolimus, or combinations thereof;
a glucocorticoid selected from the group consisting of dexamethasone acetate, dexamethasone, dexamethasone laurate, dexamethasone tert-butylacetate, dexamethasone tetrahydrophthalate, dexamethasone isonicontinate or combinations thereof;
a dose of mTOR inhibitor of about 20 to about 100 μg/cm², preferably about 25 to about 75 μg/cm² and most preferably, when the mTOR inhibitor is zotarolimus, about 35 μg/cm²;
a release rate of the mTOR inhibitor of about 50 to about 90% at about 28 days, preferably about 80% at about 28 days.
a dose of glucocorticoid of about 40 to about 200 μg/cm², preferably about 50 to about 150 μg/cm² and most preferably when the glucocorticoid is dexamethasone acetate, with a dose of about 70 μg/cm².

1. A method of treating a vascular lesion in a patient, comprising delivering to the site of the vascular lesion an implantable medical device comprising a drug reservoir layer comprising about 20 to less than 100 μg/cm² of an mTOR inhibitor and about 40 μg/cm² to less than 200 μg/cm² of a glucocorticoid, wherein the release rate of both the mTOR inhibitor and the glucocorticoid is about 50% to about 90% at about 7 to about 90 days post implant.

2. The method of claim 1, wherein the release rate of the mTOR inhibitor is about 50% to about 90% at about 28 days post implant.

3. The method of claim 1, wherein the release rate of the glucocorticoid is about 50% to about 90% at about 28 days post implant.

4. The method of claim 2, wherein the release rate of the mTOR inhibitor is about 80% at about 28 days post implant.

5. The method of claim 4, wherein the release rate of the glucocorticoid is about 80% at about 28 days post implant.

6. The method of claim 1, wherein the mTOR inhibitor is selected from the group consisting of everolimus, zotarolimus, sirolimus, sirolimus derivatives, biolimus, myolimus, novolimus, temsirolimus, merilimus, deforolimus and combinations thereof.

7. The method of claim 6, wherein the mTOR inhibitor is zotarolimus.

8. The method of claim 1, wherein the glucocorticoid is selected from the group consisting of dexamethasone and a derivative of dexamethasone that is as, or more, hydrophobic than dexamethasone.

9. The method of claim 8, wherein the dexamethasone derivative is selected from the group consisting of dexamethasone acetate, dexamethasone laurate, dexamethasone tert-butylacetate, dexamethasone tetrahydrophthalate, and dexamethasone isonicotinate.

10. The method of claim 9, wherein the glucocorticoid is dexamethasone acetate.

11. The method of claim 1, wherein the implantable medical device comprises a stent.

12. The method of claim 1, wherein the drug reservoir layer comprises a polymer or combination of polymers that exhibit a Hildebrand solubility parameter of about 7 to about 12.5 (cal/cm³)^{0.5}.

13. The method of claim 12, wherein the polymer is selected from the group consisting of poly(vinylidene fluoride-co-chlorotrifluoroethylene) (PVDF-CTFE), poly(vinylidene fluoride-co-tetrafluoroethylene) (PVDF-TFE), po)y(vinylidene fluoride-co-hexafluoropropylene-co-tetrafluoroethylene) and combinations thereof.

14. The method of claim 1, wherein the vascular lesion is selected from the group consisting of diffuse or long lesions, small vessel lesions, saphenous vein graft lesions, restenotic lesions, bifurcation lesions, ostial lesions, left main lesions, chronic total occlusions and occlusions associated with AMI or STEMI.

15. The method of claim 1, wherein the lesion is of the coronary, neurologic, carotid, aortic, renal, iliac, femoral, popliteal or tibial vasculature.

16. The method of claim 1, wherein the drug reservoir layer comprises about 25 to about 75 μg/cm² of the mTOR inhibitor.

17. The method of claim 7, wherein the drug reservoir layer comprises about 35 μg/cm² of zotarolimus.

18. The method of claim 1, wherein the drug reservoir layer comprises about 50 to about 150 μg/cm² of the glucocortidoid.

19. The method of claim 17, wherein the drug reservoir layer comprises about 70 μg/cm² of dexamethasone acetate.

20. The method of claim 1, wherein the patient is a diabetic.

21. The method of claim 1, wherein the vascular lesion is about 18 in length or longer.

22. The method of claim 1, wherein the lesion is diffuse.
